SEQUENCE LISTING (30 sequences)

SEQ ID NO: 1 - DNA, 20 bp, Artificial Sequence
  ACGT_0060 F
caaggggacaactacttctg

SEQ ID NO: 2 - DNA, 20 bp, Artificial Sequence
  ACGT_0060 R
agctaaccaagctcattttg

SEQ ID NO: 3 - DNA, 20 bp, Artificial Sequence
  ACGT_0067 F
tttgcttgattcaatgtgtt

SEQ ID NO: 4 - DNA, 20 bp, Artificial Sequence
  ACGT_0067 R
ttcaaattcaacgggaatac

SEQ ID NO: 5 - DNA, 20 bp, Artificial Sequence
  ACGT_0068 F
tgcaatattaaaggggaaaa

SEQ ID NO: 6 - DNA, 20 bp, Artificial Sequence
  ACGT_0068 R
tgcattgatatcttcgtcaa

SEQ ID NO: 7 - DNA, 21 bp, Artificial Sequence
  ACGT_0070 F
ccaaactcagaagtacaatcg

SEQ ID NO: 8 - DNA, 20 bp, Artificial Sequence
  ACGT_0070 R
atccatattcgggtcagatt

SEQ ID NO: 9 - DNA, 20 bp, Artificial Sequence
  ACGT_0071 F
attattccccatctcattcc

SEQ ID NO: 10 - DNA, 20 bp, Artificial Sequence
  ACGT_0071 R
ttcctttcattcgtcctcta

SEQ ID NO: 11 - DNA, 20 bp, Artificial Sequence
  ACGT_0072 F
gggtgtggagataatctgtc

SEQ ID NO: 12 - DNA, 20 bp, Artificial Sequence
  ACGT_0072 R
attcgatttagtttggctca

SEQ ID NO: 13 - DNA, 20 bp, Artificial Sequence
  ACGT_0078 F
ttttacaggaagtgctgagg

SEQ ID NO: 14 - DNA, 20 bp, Artificial Sequence
  ACGT_0078 R
aacataaaatggctgcaaat

SEQ ID NO: 15 - DNA, 20 bp, Artificial Sequence
  ACGT_0079 F
tatcttttggtttttgttgg

SEQ ID NO: 16 - DNA, 20 bp, Artificial Sequence
  ACGT_0079 R
agcagctatttcaggtaacg

SEQ ID NO: 17 - DNA, 20 bp, Artificial Sequence
  ACGT_0085 F
aaagttagagcaccgaaaca

SEQ ID NO: 18 - DNA, 20 bp, Artificial Sequence
  ACGT_0085 R
cgggttttcaacttaatgag

SEQ ID NO: 19 - DNA, 22 bp, Artificial Sequence
  ACGT_0086 F
ggttgttgagtttagtaatttt

SEQ ID NO: 20 - DNA, 20 bp, Artificial Sequence
  ACGT_0086 R
ttttcaacatgcattacacg

SEQ ID NO: 21 - DNA, 176 bp, Artificial Sequence
  ACGT_0060 SSR marker allele
caaggggacaactacttctgttgtatacctagtagcattattattcattataataataataataataataataataataataataataataataataataataataataataataataataatacagtaaaatgattctctaagttactattcattcaaaatgagcttggttagct

SEQ ID NO: 22 - DNA, 168 bp, Artificial Sequence
  ACGT_0067 SSR marker allele
tttgcttgattcaatgtgttaatttatatatatatatatatatatatatatatatatatatatatatatatatatatatatatatatatattaattttttgtattaattgattttatatatgtatacatacgtacacttatatattctgtattcccgttgaatttgaa

SEQ ID NO: 23 - DNA, 135 bp, Artificial Sequence
  ACGT_0068 SSR marker allele
tgcaatattaaaggggaaaagaatatatatatatatatatatatatatatatatatatatatatatatatatatatatatatatatatattaaaactttgaatctatatcataccttgacgaagatatcaatgca

SEQ ID NO: 24 - DNA, 168 bp, Artificial Sequence
  ACGT_0070 SSR marker allele
ccaaactcagaagtacaatcgaacaaagacaatatatatatatatatatatatatatatatatatatatatatatatatatatatatatatatatatatatatatatatatatatttagtggtagattggatatgaattttaaaataaaatctgacccgaatatggat

SEQ ID NO: 25 - DNA, 185 bp, Artificial Sequence
  ACGT_0071 SSR marker allele
attattccccatctcattccctcttttatatatatatatatatatatatatatatatatatatatatatatatatatatatatatatatatatatatatatatatatgggcttgagaaacaagcatcacctacaacccccaaaggccccgattccacaaacagcatagaggacgaatgaaaggaa

SEQ ID NO: 26 - DNA, 185 bp, Artificial Sequence
  ACGT_0072 SSR marker allele
gggtgtggagataatctgtcagatttcaaaaaacaaatgtagtaaagtctaatatatatatatatatatatatatatatatatatatatatatatatatatatatatatatatatatatatatatatatattaatctttgatttgatttgatttatattaatctttgagccaaactaaatcgaat

SEQ ID NO: 27 - DNA, 159 bp, Artificial Sequence
  ACGT_0078 SSR marker allele
ttttacaggaagtgctgagggtgaatttacgcatttggtcgaatgtgtgtgtgtgtatatatatatatatatatatatatatatatatatatatatatatatatatatatatatatactatattaataacaagaatacaatttgcagccattttatgtt

SEQ ID NO: 28 - DNA, 167 bp, Artificial Sequence
  ACGT_0079 SSR marker allele
tatcttttggtttttgttggtaatatatatatatatatatatatatatatatatatatatatatatatatatatatatatatatatatatatatatatatatatatatatatatatatttctacgttagtatatctaaaagggcacccgttacctgaaatagctgct

SEQ ID NO: 29 - DNA, 166 bp, Artificial Sequence
  ACGT_0085 SSR marker allele
aaagttagagcaccgaaacatagataataataataataataataataaatatatatatatatatatatatatatatatatatatatatatatatatatatatatatatatatatatatatatatatatatatatattagcgaaaagctcattaagttgaaaacccg

SEQ ID NO: 30 - DNA, 161 bp, Artificial Sequence
  ACGT_0086 SSR marker allele
ggttgttgagtttagtaatttttctattagttaggttatatatatatatatatatatatatatatatatatatatatatatatatatatatatatatatatatatatatatatatatatatacttggaacaagtataataacgtgtaatgcatgttgaaaa

(19) United States
(12) Patent Application Publication
(10) Pub. No.: US 2014/0249046 A1
(43) Pub. Date: Sep. 4, 2014

(54) SSR MARKERS FOR PLANTS AND USES THEREOF

(71) Applicant: ACGT INTELLECTUAL LIMITED, Tortola (VG)

(72) Inventors: Suan Choo Cheah, Putrajaya (MY); Sock Hwa Yong, Petaling Jaya (MY); Weng Wah Lee, Puchong (MY); Soon Joo Yap, Bandar Baru Bangi (MY); Yang Ming Poh, Seri Kambangan (MY); Ying Wah Lee, Seremban (MY)

(21) Appl. No.: 14/232,865

(22) PCT Filed: Sep. 30, 2011
(86) PCT No.: PCT/MY2011/000212

§ 371 Date: Apr. 15, 2014

(30) Foreign Application Priority Data
Jul. 15, 2011 (MY) PI2011003339

(51) Int. Cl. C12Q 1/68

(52) U.S. Cl. CPC C12Q 1/6895
USPC 506/9; 435/6.11; 536/24.33; 536/23.1

(57) ABSTRACT

Simple sequence repeat (SSR) markers identified in Jatropha curcas and useful for the molecular genotyping of plants. are described. These markers may be used for identifying allele polymorphisms, identifying identical or related plants, differentiating plants and studying genetic diversity in a population. The markers may also be used in genetic and phenotype studies using statistical methods, for example, linkage analysis, association mapping, linkage disequilibrium and the like. The information may be used for breeding and/or selection of plants.

### FIELD OF THE INVENTION

The present invention relates to the field of molecular genotyping. In particular, the invention relates to the identification and isolation of simple sequence repeat (SSR) markers and their application to genotyping.

### BACKGROUND OF THE INVENTION

Jatropha curcas (Family Euphorbiaceae), also known as physic nut, is a non-food crop oil-seed bearing tree (or large shrub) which can grow up to 5 meters. J. curcas seems to be native to central or South America. It is now grown across the tropics and sub-tropic, such as Africa and Asia. Naturally, it is cross pollinated by insects but can be propagated by cutting as well. J. curcas has never been extensively bred for productivity.

The extent of genetic diversity is a prerequisite for a crop improvement program. Morphological characterization of genetic diversity can be biased due to the strong influence of environment even on highly heritable seed traits such as average seed weight, seed protein and oil content in J. curcas. Hence, genetic information generated using neutral molecular markers (not influenced by the environment) is essential as this is more reliable and consistent. There is little information regarding the origin and the genetic diversity of J. curcas populations from different places. Thus, the identification of the genetic diversity of the germplasm will be useful to identify parental lines suitable for genetic improvement (breeding programme) and genetic mapping.

Simple sequence repeats (SSR, also known as SSRs or microsatellites) are tandem repeats of short nucleotide sequences, 2-6 bases in length, that vary in number. SSR may be amplified by the polymerase chain reaction (PCR) with two or more specific primers. After amplification, PCR products of different lengths are produced, representing allele polymorphisms. Null alleles with no amplification also occur when there are mutations within the binding site for the primer.

SSR are useful for assessing genetic diversity (Ashley et al. 2003). SSR markers are preferable because they are often codominant, highly reproducible, and frequent in most eukaryotes and reveal high allelic diversity (Mohan et al., 1997). However, according to a study by Sun et al., 2008, polymorphism was not detected among 56 Chinese J. curcas accessions using 17 SSR primers developed by FIASCO (Fast Isolation by AFLP of Sequences Containing repeat) protocol. However, it was reported in the same paper that only AFLP markers showed polymorphisms within the Chinese J. curcas accessions.

There is thus a need to provide novel tools for the genetic analysis of J. curcas for screening a population for genotyping purposes, phylogeny and also for genetic and linkage mapping.

### SUMMARY OF THE INVENTION

The present invention relates to SSR markers and primers for amplifying SSR markers. The amplified SSR markers vary in size and are polymorphic alleles. The SSR markers of the present invention may be used for molecular genotyping and/or genetic fingerprinting.

According to a first aspect, the present invention provides a method for determining the genotype of a plant sample comprising:
(i) providing DNA from the sample;
(ii) amplifying at least one polymorphic SSR marker with at least one primer pair selected from the group consisting of SEQ ID NOs: 1 and 2; 3 and 4; 5 and 6; 7 and 8; 9 and 10; 11 and 12; 13 and 14; 15 and 16; 17 and 18; and 19 and 20 or a fragment or variant thereof of each pair;
and
(iii) identifying at least one polymorphic allele of the SSR maker present in the sample.

The amplified products may be separated to identify the alleles present. Alternatively, polymorphism of a SSR marker in a sample may be identified by sequencing.

The invention also provides an isolated oligonucleotide primer for amplifying at least one SSR marker, selected from the group consisting of SEQ ID NOs: 1-20 or a fragment of variant thereof. The invention further provides an isolated oligonucleotide primer pair for amplifying at least SSR marker selected from the group consisting of SEQ ID NOs: 1 and 2; 3 and 4; 5 and 6; 7 and 8; 9 and 10; 11 and 12; 13 and 14; 15 and 16; 17 and 18 and 19 and 20 or a fragment or variant of each primer pair.

### BRIEF DESCRIPTION OF THE FIGURES

FIG. 1 illustrates an electropherogram of 8 J. curcas samples amplified using primer pair comprising SEQ ID NOs: 11 and 12.

FIG. 2 illustrates a silver stained polyacrylamide gel of 8 J. curcas samples amplified using primer pair comprising SEQ ID NOs: 11 and 12.

FIG. 3 illustrates the principle component analysis (PCA) results of 927 J. curcas samples genotyped with the ten SSR markers.

FIG. 4 illustrates the bar plot results of the K=3 simulated dataset generated using the ten SSR markers to genotype 927 J. curcas samples.

FIG. 5 illustrates a plot of ΔK=m|L“(K)I/S [L(K)] against K from the STRUCTURE cluster analysis.

### DEFINITIONS

The abbreviation “SSR” stands for “simple sequence repeat” and refers to any short sequence, that is repeated at least once in a particular nucleotide sequence. SSR may be found in both coding and non-coding areas of genomes of an organism. The term SSR may be used interchangeably with “microsatellite”. A SSR can be represented by the general formula (N₁N₂ . . . Nᵢ)ₙ, wherein N represents nucleotides A, T, C or G, i represents the number of the nucleotides in the base repeat, and n represents the number of times the base is repeated in a particular DNA sequence. The base repeat, i.e. N₁N₂ . . . Nᵢ is also referred to as a “SSR motif”. The repeating SSR motif typically may be a mono-, di-, tri- or tetra-nucleotide motif. For example (ATC)₄ refers to a tri-nucleotide. SSR are highly polymorphic, in that each SSR locus may have a number of “allelic” forms. Polymorphic SSR loci are extremely useful markers in any organism for identification, paternity testing and genetic mapping. Polymorphism is a feature of SSR which contributes to their usefulness in genotyping and/or genetic fingerprinting.

“Perfect repeat” refers to a repeated SSR motif without interruption and without adjacent repeat(s) of a different motif. However, the repeats may be “imperfect” when a repeated SSR motif is interrupted by a number of non-repeated nucleotides, such as for example in (AC)₅GCTAGT(AC)₇. An imperfect repeat may also be viewed as a repeat sequence, where some individual bases are mutated. Other possible variations of SSRs would be known to those of skill in the art. These repeats, including compound repeats, are defined by Weber (1990).

“Compound repeat” refers to a SSR that contains at least two different repeated motifs that may be separated by a stretch of non-repeated nucleotides. An example of a compound repeat is (ATC)₅(AT)₆.

“SSR locus” refers to a location on a chromosome of a SSR marker. The locus may be occupied by any one of the alleles of the SSR marker. “Allele” is one of several alternative forms of the SSR marker occupying a given locus on the chromosome.

### DETAILED DESCRIPTION OF THE INVENTION

The oligonucleotide primers and SSR markers of the present invention were obtained from J. curcas genome data. The SSR markers and isolated oligonucleotide primers may be used for distinguishing Jatropha species. In particular, the SSR markers and isolated oligonucleotide primers may be used for distinguishing J. curcas from other Jatropha species.

The SSR markers are amplified by oligonucleotide primers. Exemplary oligonucleotide primers of the present invention comprise the following sequences in Table 1.

Each of the ten primer pairs of Table 1 may be used to amplify a SSR marker from a plant sample. Each of the oligonucleotide primer pairs and/or SSR markers of the present invention reveal polymorphism in J. curcas samples.

TABLE 1
Examples of isolated oligonucleotide primers
according to the invention

| SEQ ID NO: | Primer sets ID | Forward (5′-3′) | Repeats |
|---|---|---|---|
| 1 | ACGT_0060 F* | CAA GGG GAC AAC TAC TTC TG | (ATA)25 |
| 2 | ACGT_0060 R | AGC TAA CCA AGC TCA TTT TG | |
| 3 | ACGT_0067 F* | TTT GCT TGA TTC AAT GTG TT | (TA)33 |
| 4 | ACGT_0067 R | TTC AAA TTC AAC GGG AAT AC | |
| 5 | ACGT_0068 F* | TGC AAT ATT AAA GGG GAA AA | (AT)34 |
| 6 | ACGT_0068 R | TGC ATT GAT ATC TTC GTC AA | |
| 7 | ACGT_0070 F* | CCA AAC TCA GAA GTA CAA TCG | (AT)42 |
| 8 | ACGT_0070 R | ATC CAT ATT CGG GTC AGA TT | |
| 9 | ACGT_0071 F* | ATT ATT CCC CAT CTC ATT CC | (TA)40 |
| 10 | ACGT_0071 R | TTC CTT TCA TTC GTC CTC TA | |
| 11 | ACGT_0072 F* | GGG TGT GGA GAT AAT CTG TC | (AT)40 |
| 12 | ACGT_0072 R | ATT CGA TTT AGT TTG GCT CA | |
| 13 | ACGT_0078 F* | TTT TAC AGG AAG TGC TGA GG | (TA)31 |
| 14 | ACGT_0078 R | AAC ATA AAA TGG CTG CAA AT | |
| 15 | ACGT_0079 F* | TAT CTT TTG GTT TTT GTT GG | (AT)48 |
| 16 | ACGT_0079 R | AGC AGC TAT TTC AGG TAA CG | |
| 17 | ACGT_0085 F* | AAA GTT AGA GCA CCG AAA CA | (AT)44 |
| 18 | ACGT_0085 R | CGG GTT TTC AAC TTA ATG AG | |
| 19 | ACGT_0086 F* | GGT TGT TGA GTT TAG TAA ATT T | (TA)43 |
| 20 | ACGT_0086 R | TTT TCA ACA TGC ATT ACA CG | |

*F indicates forward primers labelled with fluorescent M13 tag may be used for PCR (see also Table 4)
R indicates reverse primers.

The invention also includes a fragment or variant of an oligonucleotide primer of Table 1. A fragment or variant thereof of an oligonucleotide primer includes any oligonucleotide primer capable of amplifying a polymorphic SSR marker according to the invention. A fragment of an oligonucleotide primer may comprise a portion of SEQ ID NOs 1-20, and includes for example, a sequence of 5-19 by from an exemplified oligonucleotide of 20 bp.

A variant oligonucleotide primer need not share any overlap with SEQ ID NOs: 1-20 but merely has to be capable of amplifying a polymorphic SSR marker according to the invention. A variant oligonucleotide primer also includes any oligonucleotide primer complementary to a region flanking a polymorphic SSR marker according to the invention. As understood by the person skilled in the art, the 3′ end of a variant oligonucleotide primer for PCR may not have any mismatches to the SSR marker or a region flanking the SSR marker while the 5′ end may have mismatches.

The invention also provides a kit comprising at least one olionucleotide primer from Table 1 or a fragment or variant thereof.

Each primer pair according to Table 1 amplifies alleles of an SSR marker from J. curcas. The amplified products vary in size and represent different polymorphic alleles of the SSR marker. Accordingly, the present invention relates to a SSR marker comprising a sequence amplified by a primer pair according to the invention.

Different polymorphic alleles of the SSR marker at each of the ten loci have different sequences. The present invention includes the sequences of the different polymorphic alleles of each of the ten SSR markers. For example, each of the ten sequences below represent a particular allele of the ten SSR markers amplified by the oligonucleotide primers SEQ ID NOs: 1-20.

ACGT_0060 SSR marker allele (SEQ ID NO: 21) amplified by SEQ ID NOs: 1 and 2:
CAAGGGGACAACTACTTCTGTTGTATACCTAGTAGCATTATTATTCATTATAATAATAATAAT
AATAATAATAATAATAATAATAATAATAATAATAATAATAATAATAATAATAATAATAATACAGT
AAAATGATTCTCTAAGTTACTATTCATTCAAAATGAGCTTGGTTAGCT
ACGT_0067 SSR marker allele (SEQ ID NO: 22) amplified by SEQ ID NOs: 3 and 4:
TTTGCTTGATTCAATGTGTTAATTTATATATATATATATATATATATATATATATATATATATAT
ATATATATATATATATATATATATATTAATTTTTTGTATTAATTGATTTTATATATGTATACATAC
GTACACTTATATATTCTGTATTCCCGTTGAATTTGAA
ACGT_0068 SSR marker allele (SEQ ID NO: 23) amplified by SEQ ID NOs: 5 and 6:
TGCAATATTAAAGGGGAAAAGAATATATATATATATATATATATATATATATATATATATATAT
ATATATATATATATATATATATATATTAAAACTTTGAATCTATATCATACCTTGACGAAGATAT
CAATGCA
ACGT_0070 SSR marker allele (SEQ ID NO: 24) amplified by SEQ ID NOs: 7 and 8:
CCAAACTCAGAAGTACAATCGAACAAAGACAATATATATATATATATATATATATATATATATA
TATATATATATATATATATATATATATATATATATATATATATATATATATTTAGTGGTAGATTG
GATATGAATTTTAAAATAAAATCTGACCCGAATATGGAT
ACGT_0071 SSR marker allele (SEQ ID NO: 25) amplified by SEQ ID NOs: 9 and 10:
ATTATTCCCCATCTCATTCCCTCTTTTATATATATATATATATATATATATATATATATATATAT
ATATATATATATATATATATATATATATATATATATATATATGGGCTTGAGAAACAAGCATCAC
CTACAACCCCCAAAGGCCCCGATTCCACAAACAGCATAGAGGACGAATGAAAGGAA
ACGT_0072 SSR marker allele (SEQ ID NO: 26) amplified by SEQ ID NOs: 11 and 12:
GGGTGTGGAGATAATCTGTCAGATTTCAAAAAACAAATGTAGTAAAGTCTAATATATATATAT
ATATATATATATATATATATATATATATATATATATATATATATATATATATATATATATATATAT
ATTAATCTTTGATTTGATTTGATTTATATTAATCTTTGAGCCAAACTAAATCGAAT
ACGT_0078 SSR marker allele (SEQ ID NO: 27) amplified by SEQ ID NOs: 13 and 14:
TTTTACAGGAAGTGCTGAGGGTGAATTTACGCATTTGGTCGAATGTGTGTGTGTGTATATAT
ATATATATATATATATATATATATATATATATATATATATATATATATATATATACTATATTAATA
ACAAGAATACAATTTGCAGCCATTTTATGTT
ACGT_0079 SSR marker allele (SEQ ID NO: 28) amplified by SEQ ID NOs: 15 and 16:
TATCTTTTGGTTTTTGTTGGTAATATATATATATATATATATATATATATATATATATATATATA
TATATATATATATATATATATATATATATATATATATATATATATATATATATTTCTACGTTAGTA
TATCTAAAAGGGCACCCGTTACCTGAAATAGCTGCT
ACGT_0085 SSR marker allele (SEQ ID NO: 29) amplified by SEQ ID NOs: 17 and 18:
AAAGTTAGAGCACCGAAACATAGATAATAATAATAATAATAATAATAAATATATATATATATAT
ATATATATATATATATATATATATATATATATATATATATATATATATATATATATATATATATAT
ATATATTAGCGAAAAGCTCATTAAGTTGAAAACCCG
ACGT_0086 SSR marker allele (SEQ ID NO: 30) amplified by SEQ ID NOs: 19 and 20:
GGTTGTTGAGTTTAGTAATTTTTCTATTAGTTAGGTTATATATATATATATATATATATATATAT
ATATATATATATATATATATATATATATATATATATATATATATATATATATATATACTTGGAAC
AAGTATAATAACGTGTAATGCATGTTGAAAA

Accordingly, the invention comprises a sequence selected from SEQ ID NO: 21-30 or a fragment or variant thereof. For example, the variant of the SSR marker is a polymorphic variant (or allele). In particular, the polymorphic variant comprises either the repeating SSR motif (TA)ₙ or (TAA)ₙ,

The number of polymorphic alleles of each SSR marker identified is shown in Table 2.

TABLE 2
SSR marker and the number of polymorphic alleles identified

| | No. of polymorhic | | Allele size |
|---|---|---|---|
| SSR Marker | alleles | Allele sizes (bp) | range (bp) |
| ACGT_0060 | 4 | 191, 194, 197, 218 | 191-218 |
| ACGT_0067 | 12 | 160, 172, 174, 176, | 160-210 |
| | | 178, 182, 184, 186, | |
| | | 188, 190, 200, 210 | |
| ACGT_0068 | 6 | 144, 148, 150, 152, | 144-156 |
| | | 154, 156 | |
| ACGT_0070 | 10 | 160, 166, 176, 178, | 160-190 |
| | | 180, 182, 184, 186, | |
| | | 188, 190 | |
| ACGT_0071 | 11 | 143, 160, 195, 197, | 143-237 |
| | | 199, 201, 203, 207, | |
| | | 233, 235, 237 | |
| ACGT_0072 | 17 | 153, 157, 177, 181, | 153-217 |
| | | 185, 187, 195, 197, | |
| | | 199, 201, 203, 205, | |
| | | 207, 209, 211, 215, | |
| | | 217 | |
| ACGT_0078 | 14 | 165, 169, 175, 177, | 165-209 |
| | | 179, 181, 183, 185, | |
| | | 187, 189, 191, 199, | |
| | | 207, 209 | |
| ACGT_0079 | 19 | 108, 112, 126, 128, | 108-204 |
| | | 130, 146, 152, 166, | |
| | | 168, 170, 174, 176, | |
| | | 182, 184, 186, 190, | |
| | | 200, 202, 204 | |
| ACGT_0085 | 12 | 120, 124, 160, 162, | 120-188 |
| | | 166, 170, 176, 178, | |
| | | 180, 182, 184, 188 | |
| ACGT_0086 | 10 | 127, 141, 165, 173, | 127-197 |
| | | 175, 177, 179, 181, | |
| | | 195, 197 | |

The number of polymorphic alleles identified for each SSR marker in Table 2 is not exhaustive. For example, the number of polymorphic alleles identified may depend on the analysis method. In particular, the resolution and/or discrimination of the analysis method may affect the number of polymorphic alleles identified. Using different flanking primers in the PCR amplification may also identify additional polymorphic alleles for each SSR marker. Accordingly, the invention comprises any polymorphic allele of the SSR markers, including polymorphic alleles of the SSR markers not listed in Table 2.

The polymorphic alleles of each SSR markers in J. curcas may be identified by PCR using the respective PCR primers. Following PCR amplification, the amplified products may be separated to identify the polymorphic alleles present in the sample. Standard separation methods may be used. For example, capillary electrophoresis or gel electrophoresis may be used to separate the amplified products. With gel electrophoresis, agarose, native or denaturing polyacrylamide gel electrophoresis may be used for separating the amplified products. Accordingly, the method of the invention comprises amplifying at least one SSR marker for identifying allele polymorphisms. The method may comprise amplifying two or more of the SSR markers with the respective primer pairs. The method may comprise amplifying any two, three, four, five, six, seven, eight or nine SSR markers with the respective primer pairs for identifying allele polymorphisms. According to a particular embodiment, all ten markers are analysed for polymorphisms. Each amplification reaction may be carried out separately or amplification of two or more SSR markers may be carried out together in a single reaction (multiplex PCR).

In particular, the method comprises:

amplifying each of the ten SSR markers with the corresponding primer pairs; and identifying at least one polymorphic allele from each of the ten amplified products in the sample.

The molecular genotyping and/or genetic fingerprinting method of the invention may be used for:

(i) identifying identical or related plant genotypes in a population;

(ii) differentiating plant variants in a population; or

(iii) studying genetic diversity in a population.

For example, related plant genotypes may be classified. Identifying related plant genotypes also includes paternity testing.

Although the SSR markers of the present invention are obtained from J. curcas, they are applicable to molecular genotyping of any plant, in particular oil producing plant. Examples of oil producing plant include but are not limited to, Jatropha, oil palm, soy bean and the like. Examples of Jatropha include other Jatropha species as well as J. curcas. In particular, the Jatropha species is J. curcas L.

According to another embodiment, the invention provides a method for distinguishing Jatropha curcas, comprising the steps of:

(i) providing DNA from a plant sample;

(ii) amplifying at least one polymorphic SSR marker with at least one primer pair selected from the group consisting of SEQ ID NOs: 1 and 2; 3 and 4; 5 and 6; 7 and 8; 9 and 10; 11 and 12; 13 and 14; 15 and 16; 17 and 18 and 19 and 20 or a fragment or variant of each pair; and

(iii) identifying at least one polymorphic allele corresponding to a J. curcas allele in the sample.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Russel (2001).

### Example 1

### PCR Amplification of SSR Markers using the Oligonucleotide Primers of Table 1 and Detection by Capillary Electrophoresis

Reaction mixes for amplification of the SSR markers in 10 μl consisted of 2 mM MgCl₂, 1× PCR buffer, 0.2 mM of dNTP mixes, 250 nM of each primer of the primer pair, 1 unit of Taq polymerase and 20 ng of DNA. The cycling conditions were denaturation 94° C., 5 min, 5 cycles of 94° C., 30 sec; 62° C., 30 sec (decreasing by 2° C. to 52° C.); 25 cycles of 94° C., 30 sec; 52° C., 30 sec, 72° C., 30 sec; a further 72 C for 7 min and hold at 10° C. The amplification was performed in a 96-Well GeneAmp® PCR System 9700. Other reaction mixes, cycling conditions and thermal cyclers with a heated lid may also be used.

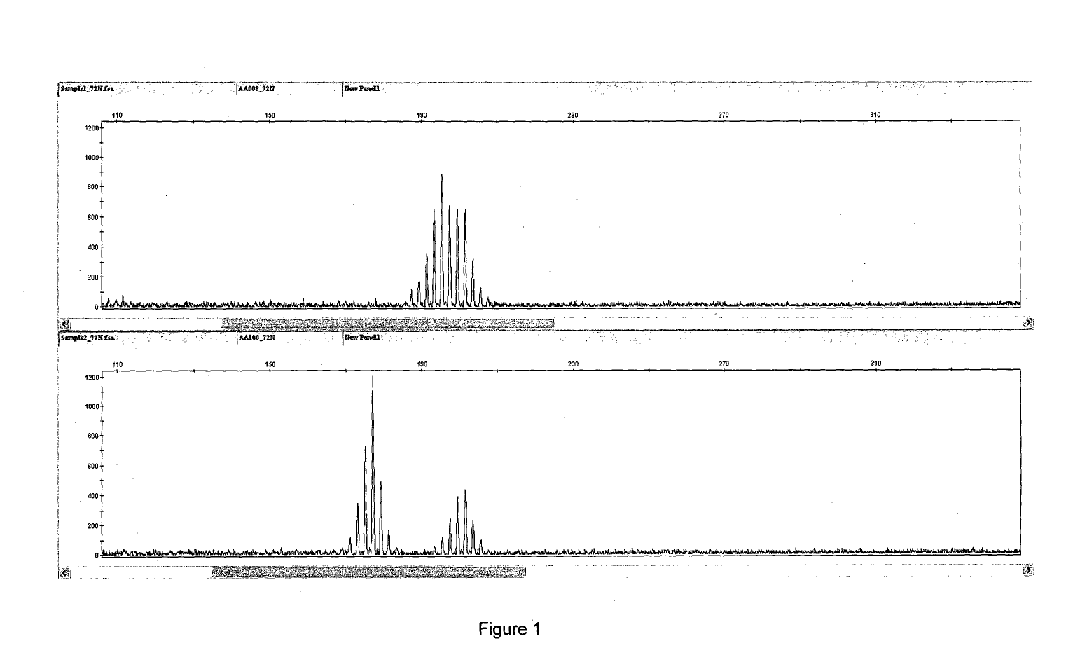

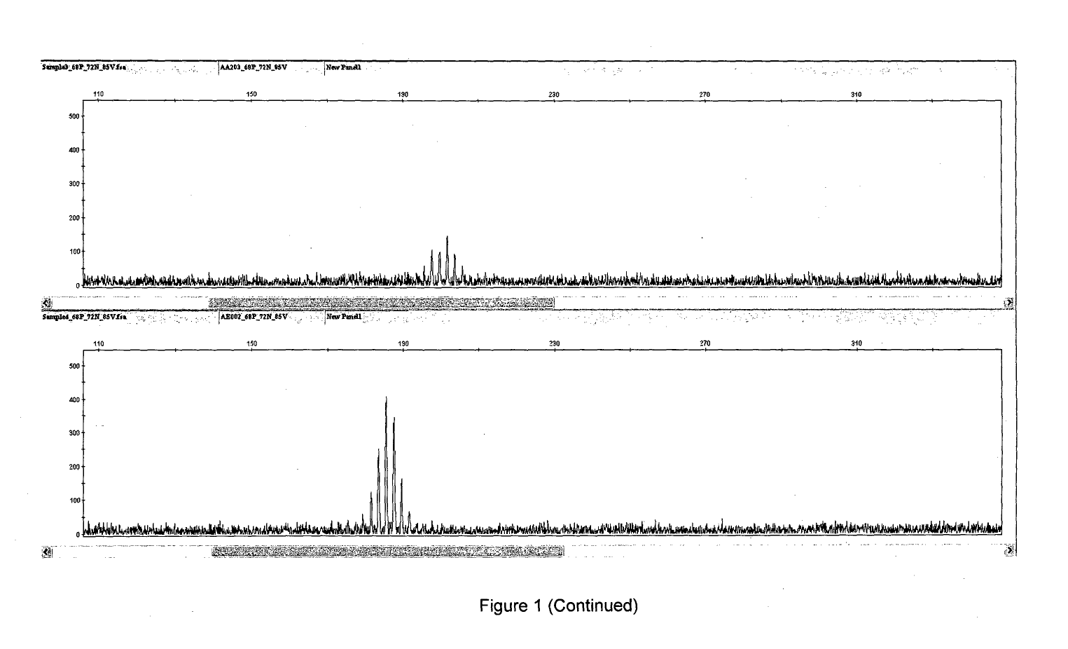

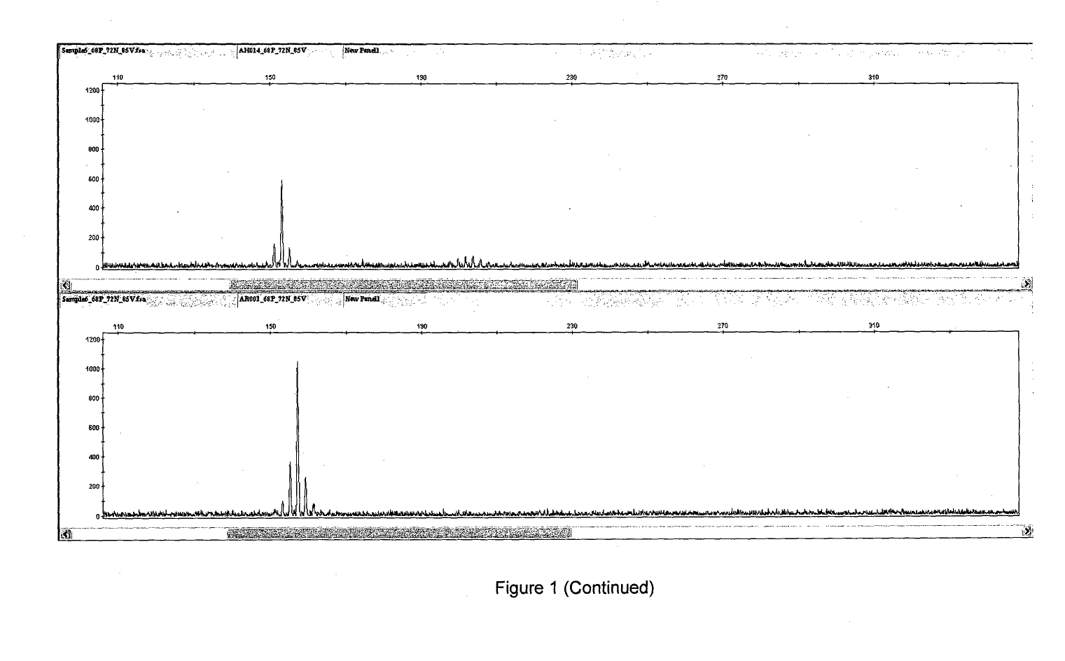

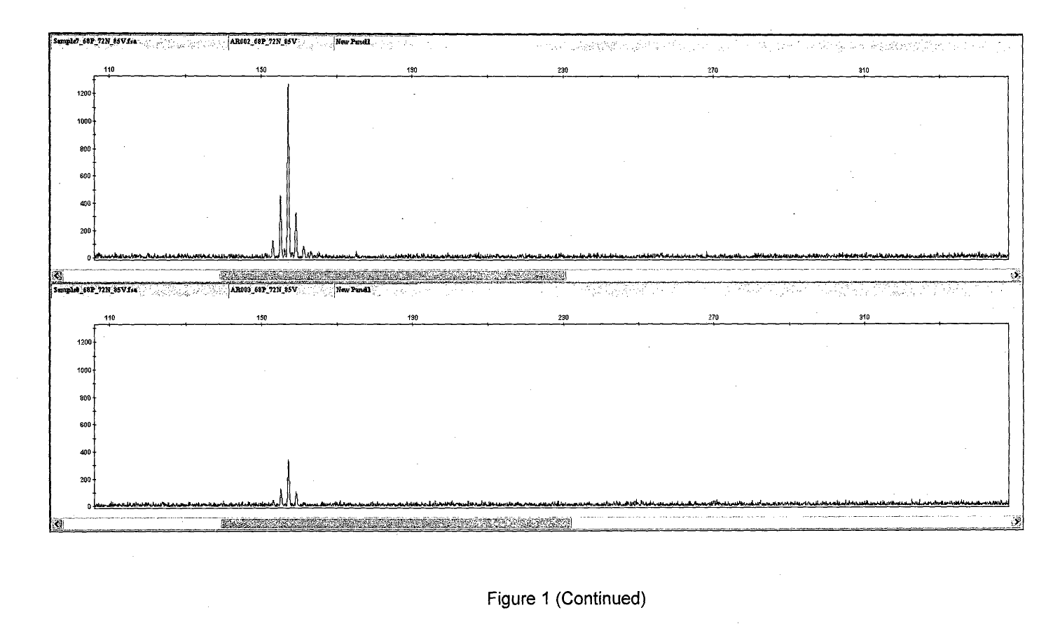

For capillary electrophoresis, fluorescent labelled primers were used to amplify the products. The amplified products were analysed by capillary electrophoresis. The sizes are determined with a standard. Samples and size standards (GeneScan 600LIZ) were heated and loaded into an Applied Biosystems ABI3730xl Genome Analyzer, a fluorescence based sequencer, according to the manufacturer's instructions. The output electropherogram will show peaks corresponding to the size of the amplified products. Scoring was performed with the Applied Biosystem GeneMapper software. FIG. 1 illustrates a capillary electropherogram of the amplification of the marker ACGT_{—}0072 SSR marker using the primer pairs SEQ ID NOs: 11 and 12, showing 5 polymorphic alleles from 8 samples. A total of 17 alleles was identified for the ACGT 0072 SSR marker (Table 2)

Alternatively, any other suitable fluorescence based sequencer, size standard and scoring method may also be used.

### Example 2

### Polyacrylamide Gel Electrophoresis (PAGE)

PCR were carried out using the same conditions as in Example 1. Polyacrylamide gel electrophoresis was used to separate the amplified products.

Amplified PCR products were heated with conventional formamide loading dye and 10 μL of each sample are separated in 7% polyacrylamide gels (1 mm thick and 25 cm long) for 6 hours at 450 volts. Table 3 shows an example of a polyacrylamide gel formulation.

TABLE 3
Formulation for polyacrylamide

| | | |
|---|---|---|
| 17.5 | ml | stock acrylamide solution (19 g |
| | | acrylamide, 1 g bisacrylamide, in 100 mL water) |
| 10 | ml | 5X TBE (1X TBE = 0.09M tris borate, |
| | | 0.002M EDTA) |
| 22.5 | ml | water |
| 220 | μl | 10% ammonium persulfate (10% APS) |
| 20 | μL | TEMED |

10% APS and TEMED were added for polymerisation.

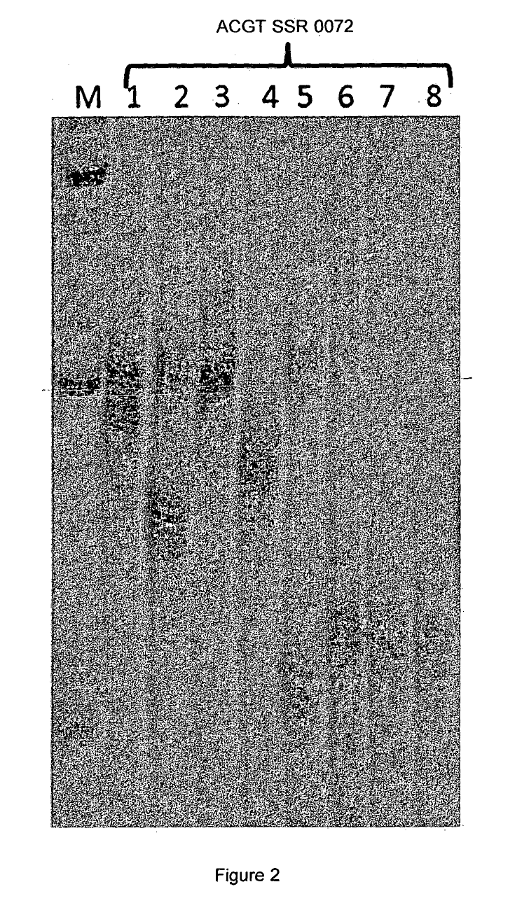

After electrophoresis, separated amplified products were visualised by silver staining or ethidium bromide staining. Detection may also be by conventional autoradiography if the primers were radiolabelled. FIG. 2 illustrates a silver stained polyacrylamide gel of 8 samples amplified using primer pair comprising SEQ ID NOs: 11 and 12. The result is similar to the capillary electrophoresis where 5 alleles were detected in the 8 samples.

### Example 3

### Cluster Analysis

Genotyping of 927 samples was carried out using the ten SSR markers according to the present invention. For each marker, PCR was carried out in 10 μl volume using the PCR mix shown in Table 4.

TABLE 4
PCR mix

| PCR mix | 10 μl PCR |
|---|---|
| H20 | 1.69 |
| 10X PCR Buffer | 1.00 |
| 50 mM MgCl2 | 0.40 |
| 10 mM dNTP | 0.20 |
| Primer mix F:R | 0.05 |
| (25 μM:50 μM) | |
| M13 tagged fluorescent | 0.50 |
| forward primer (2.5 μM)* | |
| Taq 5 u/μl | 0.16 |
| DNA (3 ng/μl) | 6.00 |
| Total volume | 10.00 |

*indicates M13 tagged fluorescent forward primer (The tagged fluorescent forward primers are also indicated in Table 1 with *).

Samples and size standards (GeneScan 600LIZ) were heated and loaded into an Applied Biosystems ABI3730xl Genome Analyzer, a fluorescence based sequencer, according to the manufacturer's instructions. Scoring was performed with the Applied Biosystem GeneMapper software. Genetic analysis was performed using NTSYSpc (Rohlf 1998) and STRUCTURE (Pritchard et al., 2010).

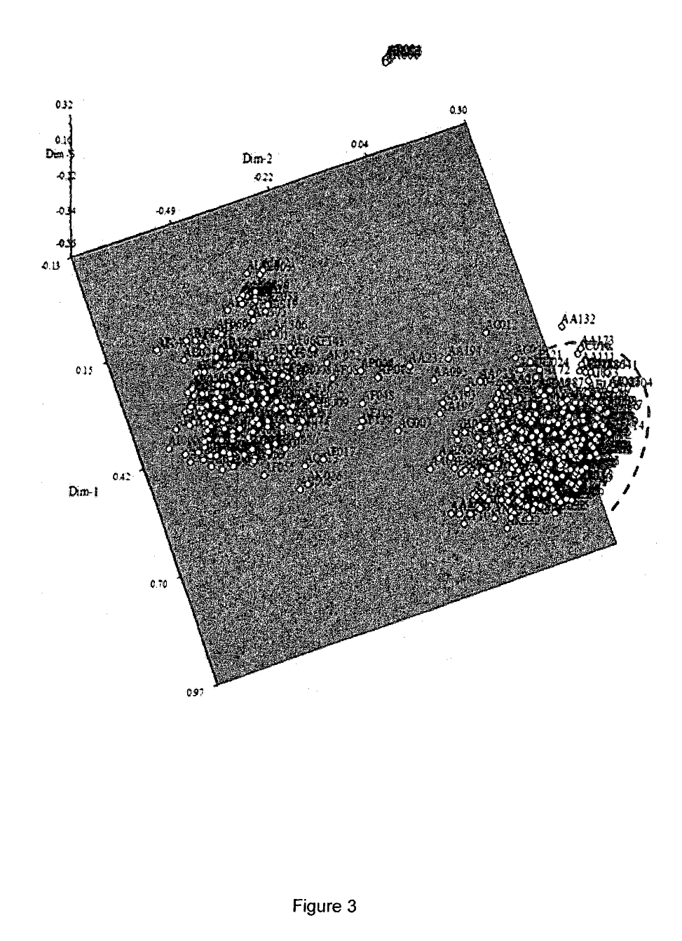

The results of the principle component analysis (PCA) by NTSYSpc for the 927 samples using the 10 SSR markers showed that there are three main clusters of J. curcas (FIG. 3).

Cluster analysis using STRUCTURE was performed (Pritchard et al., 2010) to infer population structure and assign individuals to clusters. With STRUCTURE, a model in which there are K clusters is assumed.

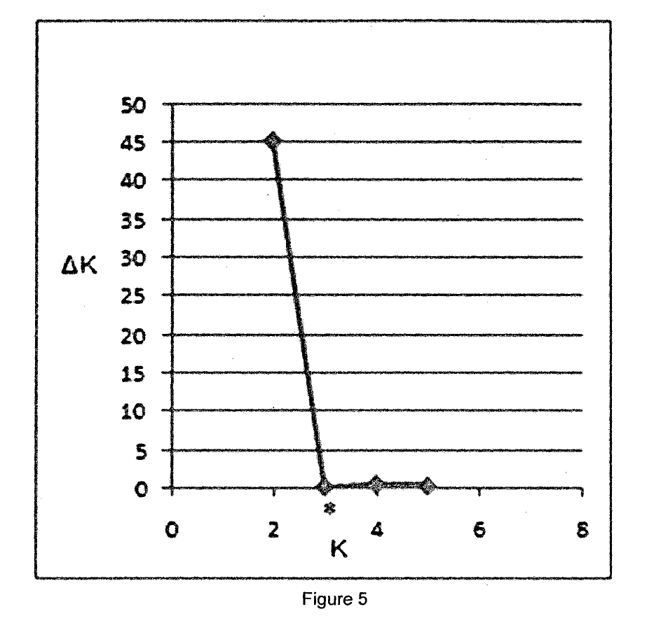

Cluster analysis was performed with burn in-lengths and Markov Chain Monte Carlo (MCMC) algorithm repetitions of 50000, 90000 and 100000 each to achieve stable results given a K (cluster), where the mean log probability of the data LnP(D) (also referred to as L(K)) is the first least difference within the set indicating the right K value (or number of clusters) (refer to FIG. 5, which indicated K=3 and Evanno et.a1.2005). Burn-in lengths and MCMC repetitions of 100000 were found to be sufficient in the analysis. From FIG. 5, which plots ΔK=m|L“(K)I/S [L(K)] against K, K was estimated to be 3 (based on the method as described in Evanno et aL, 2005). As observed, the modal value of this distribution is the true K, as illustrated by the asterisk * or the uppermost level of the structure, here 3. Importantly, it was found that the K value obtained from NTSYSpc and STRUCTURE correlate with each other.

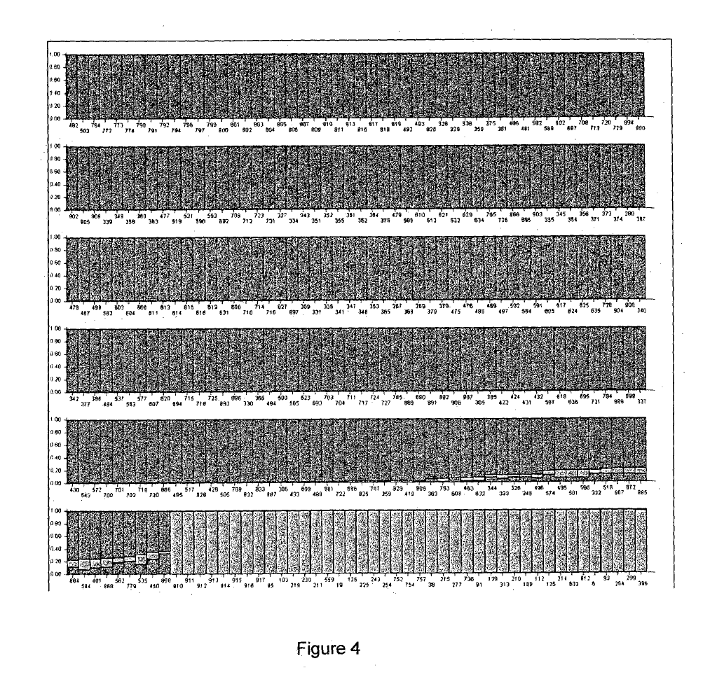

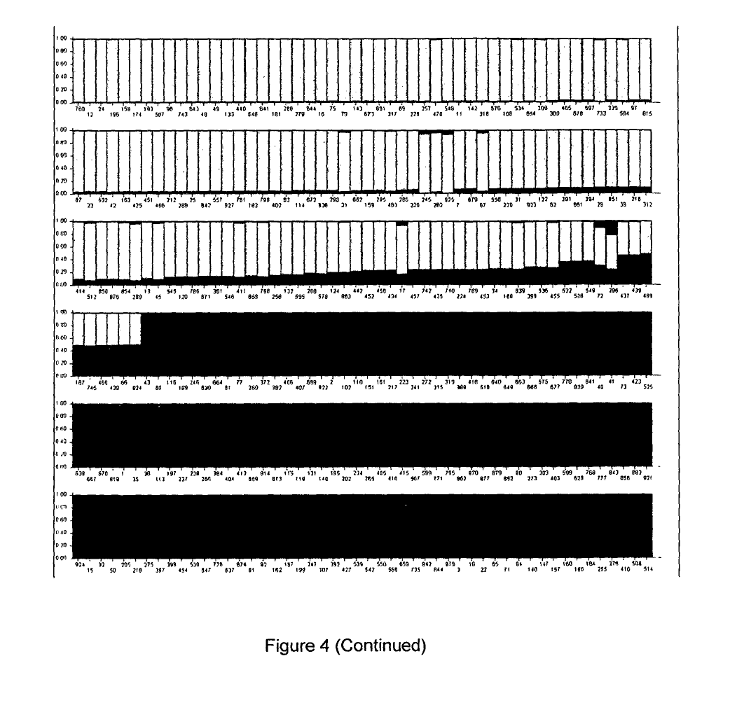

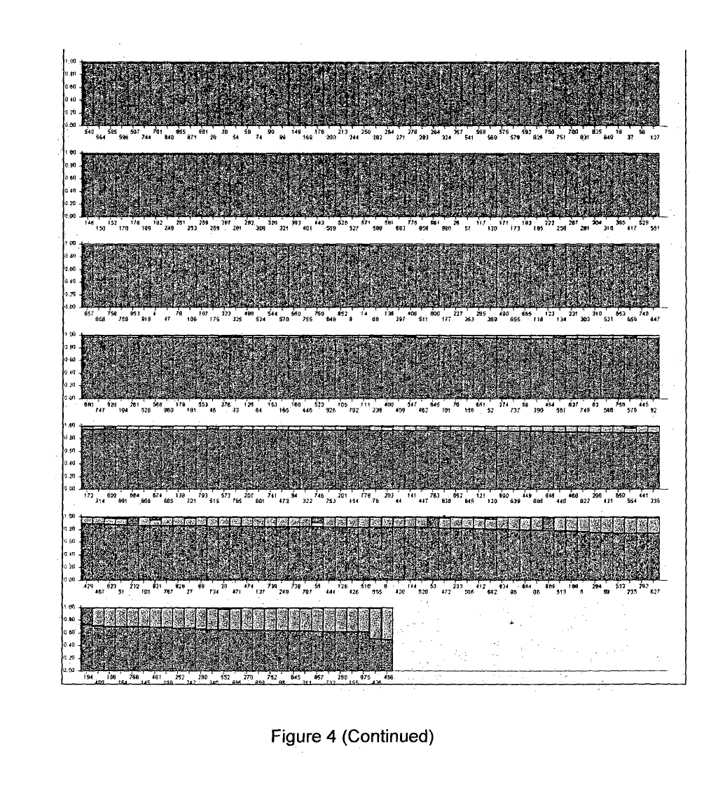

FIG. 4 illustrates the bar plot results of the K=3 simulated dataset generated using the ten SSR markers to genotype 927 J. curcas samples. Instead of LnP(D) or L(K), STRUCTURE also presents the cluster graphically as in FIG. 4. The vertical axis represents the individual's estimated membership fractions or contribution of parental origin (cluster) and the horizontal axis is the individual plants sorted according to the parental origin (cluster). Since K=3, the three clusters represented by three different shades was observed. Some individual plants may have only one parental origin (represented by a single cluster shade). Others have crosses between the parental lines (clusters) and thus have multiple shades indicating contribution of each parental cluster in the individual plants.

### Example 4

### Association Analysis

The SSR markers of the invention may be employed in genetic and phenotype studies using statistical methods. Examples of these statistical methods include linkage analysis, association mapping, linkage disequilibrium and the like. The level at which these SSR markers and genetic regions/sequences are co-inherited may be measured by linkage analysis. For example, a collection of plants exhibiting variation for a particular trait of interest may be used as the mapping population. Screening this population for the SSR markers of the present invention may be carried out to identify associations between the markers and traits of interest, the extent of linkage disequilibrium among them, genetic variations among individuals, heterozygosity and homozygosity of individual plants. In the case the markers are found to be able to characterize an individual plant or a group of plant of traits of interest, these markers may be used as a tool to screen other plants and population of plants with genetic potential to carry the trait of interest. Accordingly, the SSR markers may be for genetic mapping, analysing relationships, calculating the genetic distance between plants, identifying varieties, evaluating the purity of varieties, identifying hybrids, non-curcas species and plant breeding (to produce seeds and planting materials). The information gained from these markers can be used to determine if a plant carries a trait of interest or if a plant is sufficiently similar or if a plant is sufficiently different for breeding purposes, and selection of optimal plants for breeding, predicting plant traits and generation of distinct cultivars.

For example, linkage or association analysis may be performed using TASSEL (Bradbury et al., 2007), SPAGeDI (Hardy et al., 2002) and STRUCTURE (Falush et al., 2003 and Rosenberg et al., 2002). Any other suitable method for linkage or association analysis may also be performed. Tassel analysis may depend on or utilize external programs to support some of the calculation. In this instance we use SPAGeDI and STRUCTURE to determine the cluster and population in Jatropha to calculate the p-value.

Five traits were analysed with each of the ten SSR markers: Bunch number per month, fresh bunch weight (g/mth) per month, fruit number per month, girth growth per month and plant height growth rate using TASSEL. Tables 5a to 5e illustrate the association analysis of the ten SSR markers to the five traits. As observed, the five traits have different association profiles to the ten SSR markers. With TASSEL analysis, the association is inversely related to the p-value, the lower the p-value, the higher the correlation between the marker and the trait. Accordingly, the markers are arranged in descending order of association with the trait in each of Tables 5a to 5e.

Table 5 Association Analysis of 5 Phenotypes to the Ten SSR Markers using TASSEL and Correlation Analysis (R²).

TABLE 5a
Marker link to trait: Bunch number per month

| | TASSEL_GLM | TASSEL_MLM |
|---|---|---|
| SSR Marker | P Value | P Value |
| ACGT_0070 | 2.20E−03 | 5.51E−02 |
| ACGT_0078 | 1.12E−02 | 3.51E−01 |
| ACGT_0079 | 1.15E−01 | 2.72E−01 |
| ACGT_0086 | 4.43E−02 | 7.18E−01 |
| ACGT_0085 | 7.94E−02 | 8.82E−02 |
| ACGT_0068 | 9.60E−03 | 1.60E−02 |
| ACGT_0067 | 6.27E−02 | 5.21E−02 |
| ACGT_0071 | 9.80E−02 | 5.61E−01 |
| ACGT_0072 | 7.17E−01 | 7.82E−01 |
| ACGT_0060 | 3.16E−01 | 3.31E−01 |

TABLE 5b
Marker link to trait: Fresh bunch weight per month, g/mth

| | TASSEL_GLM | TASSEL_MLM |
|---|---|---|
| SSR marker | P Value | P Value |
| ACGT_0070 | 1.70E−03 | 6.82E−01 |
| ACGT_0085 | 1.21E−02 | 1.10E−02 |
| ACGT_0078 | 3.07E−02 | 1.61E−01 |
| ACGT_0079 | 3.67E−01 | 4.84E−01 |
| ACGT_0068 | 1.02E−02 | 5.90E−02 |
| ACGT_0071 | 5.05E−02 | 3.83E−01 |
| ACGT_0067 | 5.08E−02 | 8.41E−01 |
| ACGT_0086 | 2.45E−01 | 4.12E−01 |
| ACGT_0072 | 8.33E−01 | 1.76E−02 |
| ACGT_0060 | 9.00E−01 | 7.76E−01 |

TABLE 5c
Marker link to trait: Fruit number per month

| | TASSEL_GLM | TASSEL_MLM |
|---|---|---|
| SSR marker | P Value | P Value |
| ACGT_0070 | 9.40E−03 | 1.81E−01 |
| ACGT_0085 | 2.28E−02 | 2.69E−02 |
| ACGT_0079 | 2.71E−01 | 3.62E−01 |
| ACGT_0067 | 3.92E−02 | 3.71E−02 |
| ACGT_0078 | 1.89E−01 | 5.69E−01 |
| ACGT_0068 | 2.94E−02 | 2.52E−02 |
| ACGT_0086 | 3.53E−01 | 6.72E−01 |
| ACGT_0071 | 1.60E−01 | 4.12E−01 |
| ACGT_0072 | 8.94E−01 | 8.57E−01 |
| ACGT_0060 | 5.72E−01 | 4.91E−01 |

TABLE 5d
Marker link to trait: Girth growth rate

| | TASSEL_GLM | TASSEL_MLM |
|---|---|---|
| SSR marker | P Value | P Value |
| ACGT_0078 | 5.29E−10 | 4.30E−03 |
| ACGT_0079 | 1.22E−07 | 6.21E−04 |
| ACGT_0072 | 6.32E−06 | 1.40E−01 |
| ACGT_0067 | 2.28E−04 | 3.33E−02 |
| ACGT_0086 | 2.50E−02 | 3.73E−01 |
| ACGT_0068 | 1.50E−03 | 5.00E−03 |
| ACGT_0071 | 1.26E−02 | 5.76E−01 |
| ACGT_0085 | 6.42E−02 | 1.38E−01 |
| ACGT_0070 | 2.18E−01 | 3.86E−01 |
| ACGT_0060 | 1.60E−03 | 4.89E−01 |

TABLE 5e
Marker link to trait: Plant height growth rate

| | TASSEL_GLM | TASSEL_MLM |
|---|---|---|
| SSR marker | P Value | P Value |
| ACGT_0078 | 9.89E−09 | 1.33E−02 |
| ACGT_0067 | 4.08E−07 | 5.56E−05 |
| ACGT_0079 | 0.000196 | 2.62E−02 |
| ACGT_0071 | 0.0022 | 2.15E−01 |
| ACGT_0072 | 0.0421 | 9.26E−01 |
| ACGT_0085 | 0.0136 | 1.05E−02 |
| ACGT_0070 | 0.1215 | 2.62E−01 |
| ACGT_0068 | 0.0061 | 2.56E−02 |
| ACGT_0086 | 0.3004 | 8.54E−01 |
| ACGT_0060 | 0.0339 | 6.58E−01 |

General linear model (GLM) is a model that uses linear relationship between the genotype and the phenotype to access the association. Mixed Linear Model (MLM) is a model that considers also contribution of the linage (cluster) that was derived from the same data set before assessing the association. Accordingly, the GLM and MLM algorithms calculate association with different assumptions and both give a p-value, which reflect (the strength) of association,

MLM is generally more accurate in assessing the association in a mixed population, and GLM is more accurate if the population is pure line (1 breed or 1 genetic cluster). In the case of Jatropha, GLM analysis can be viewed as species associated markers, while MLM analysis can be viewed as subpopulation associated markers. The significance of association is relative. P-value of random and unassociated phenotype to marker is approximately 0.5 (or 5E-01) and higher. Any value below 0.5 is statistically considered associated, or has some contribution to the phenotype or trait. However, to increase the certainty of association, p-value of 0.05 (or 5E-02) is used as a higher stringency standard, where lower than this value is considered significant. The lower the p-value, the stronger the association indicated.

Frequently both GLM and MLM are interpreted together to support an association. Consider the case with the marker link to trait: Plant height growth rate (Table 5e), the marker ACGT_{—}0067 showed low p-values, 4.08E-07 (GLM) and 5.56E-05 (MLM). Both algorithms suggest that the marker ACGT_{—}0067 may be used to select for height growth rate characteristics (high growth or low growth rate) of the plant in the species and subpopulation level.

Any other trait of interest may be analysed for association with the SSR markers of the present invention.

### REFERENCES

Ashley et al., (2003) Theoretical and Applied Genetics.,107:1201-1207

Bradbury et al., (2007) Bioinformatics, 23(19):2633-2635.

Evanno et al., (2005) Molecular Ecology, 14:2611-2620.

Falush et al., (2003) Genetics, 164:1567-1587.

Rosenberg et al., (2002) Science, 298:2381-2385

Hardy et al., (2002) Molecular Ecology Notes, 2(4):618-620.

Mohan et al., (1997) Molecular Breeding, 3:87-103

Prtichard et al., (2010) Documentation for structure software: Version 2.3, http://pritch.bsd.uchicago.edu/software/structure22/readme.pdf

Rohif (1998) NTSYSpc Numerical and Mutlivariate Analysis System Version 2.0 User Guide, Exeter software.

Sambrook and Russel, (2001). Molecular Cloning: A Laboratory Manual, Cold Springs Harbor Laboratory, New York.

Sun et al., (2008) Crop Science, 48:1865-1871.

Weber (1990) Genomics, 7:524-530.

1. A method for determining the genotype of a plant sample comprising:
(i) providing DNA from the sample;
(ii) amplifying at least one polymorphic SSR marker with at least one primer pair selected from the group consisting of SEQ ID NOs: 1 and 2; 3 and 4; 5 and 6; 7 and 8; 9 and 10; 11 and 12; 13 and 14; 15 and 16; 17 and 18 and 19 and 20 or a fragment or variant of each pair; and
(iii) identifying at least one polymorphic allele in the sample.

2. The method according to claim 1, wherein step (ii) comprises amplifying two or more of the SSR markers with the corresponding primer pairs.

3. The method according to claim 1, wherein step (ii) comprises amplifying each of the ten SSR markers with the corresponding primer pair; and step (iii) comprises identifying at least one polymorphic allele from each of the ten SSR markers in the sample.

4. The method according to claims 1, wherein step (iii) comprises separating the amplified products to identify the polymorphic allele or sequencing to identify the polymorphic allele.

5. The method according to claims 1, for identifying allele polymorphisms.

6. The method according to claims 1, for identifying identical or related plant genotypes in a population.

7. The method according to claims 1, for differentiating plant variants in a population.

8. The method according to claims 1, for studying genetic diversity in a population.

9. The method according to claims 1, wherein the plant comprises an oil producing plant.

10. The method according to claims 1, wherein the plant comprises Jatropha, oil palm or soy bean.

11. The method according to claims 1, wherein the plant comprises Jatropha curcas.

12. A method for distinguishing Jatropha curcas, comprising the steps of:
(i) providing DNA from a plant sample;
(ii) amplifying at least one polymorphic SSR marker with at least one primer pair selected from the group consisting of SEQ ID NOs: 1 and 2; 3 and 4; 5 and 6; 7 and 8; 9 and 10; 11 and 12; 13 and 14; 15 and 16; 17 and 8 and 19 and 20 or a fragment or variant of each pair; and
(iii) identifying at least one polymorphic allele corresponding to a J. curcas allele in the sample.

13. An isolated oligonucleotide primer for amplifying at least one SSR marker, comprising a sequence selected from the group consisting of SEQ ID NOs: 1 -20 or a variant thereof.

14. An isolated oligonucleotide primer pair for amplifying at least one SSR marker, selected from the group consisting of SEQ ID NOs: 1 and 2; 3 and 4, 5 and 6, 7 and 8, 9 and 10, 1 and 12, 13 and 14, 15 and 16, 17 and 18 and 19 and 20 or a fragment or variant of each pair.

15. An isolated SSR marker amplified by a primer pair according to claim 13.

16. An isolated SSR marker, comprising a sequence selected from SEQ ID NOs: 21-30 or a variant thereof

17. The isolated SSR marker according to claim 16, wherein the variant comprises a polymorphic variant.

18. The isolated SSR marker according to claim 17, wherein the polymorphic variant comprises either the repeating SSR motif (TA)n or (TAA)n.

19. A kit comprising at least one isolated oligonucleotide primer according to claim 13.
